(19) United States
(12) Patent Application Publication
(10) Pub. No.: US 2021/0045972 A1
(43) Pub. Date: Feb. 18, 2021

(54) PILL DISPENSER DEVICE, SYSTEM, AND METHOD

(71) Applicant: MODOSCRIPT, LLC, Randolph, NJ (US)

(72) Inventor: David Zuleta, Mine Hill, NJ (US)

(21) Appl. No.: 16/964,961

(22) PCT Filed: Jan. 26, 2019
(86) PCT No.: PCT/IB2019/000088

§ 371 Date: Jul. 24, 2020

Related U.S. Application Data
(60) Provisional application No. 62/621,931, filed on Jan. 25, 2018.

(51) Int. Cl. A61J 7/04; A61J 7/00; G16H 20/13

(52) U.S. Cl. CPC A61J 7/0427; A61J 7/0076; A61J 2200/70; A61J 2200/30; A61J 2205/60; G16H 20/13

(57) ABSTRACT

A pill dispensing system includes a pill dispenser, which includes a body, a dispensing port in the body to dispense medicine from the body, and a computer processor supported by the body. In an embodiment, pill dispensing system includes a dispensing system platform and a pill dispenser configured to communicate with the dispensing system platform. The dispensing system platform includes an administrator module having a central database and an administrator portal configured for system administrator access. A pill dispenser key device includes a body housing a processor, a memory, and a communication element, the processor, the memory, and the communication element configured to communicate a digital key to a pill dispenser.

### REFERENCE TO RELATED APPLICATIONS

This application claims one or more inventions which were disclosed in Provisional Application No. 62/621,931, filed Jan. 25, 2018, entitled “Pill-safe Monitoring Digital Health System”. The benefit under 35 USC § 119(e) of the United States provisional application is hereby claimed, and the aforementioned application is hereby incorporated herein by reference.

### BACKGROUND OF THE INVENTION

### Field of the Invention

The invention pertains to the field of pill dispensers, and more particularly, to a tamper-proof pill dispenser apparatus and system.

### Description of Related Art

A variety of pill dispensers exist to dispense medicine in the form of pills, capsules, tablets, and others (all herein referred to as “pills”). Many have mechanisms that make it difficult for infants, toddlers, and small children to access the potentially dangerous medicine inside. Other pill dispensers have schedules to help individuals stay on schedule taking the medicine. Still other pill dispensers attempt to prevent access by keeping the dispenser locked until it is deemed to be time to ingest the medicine, at which time the dispenser unlocks, allowing an individual access to the medicine. None of these pill dispensers prevents unfettered access to the stash of pills inside the pill dispenser, which can be dangerous and problematic with patients or individuals who are addicted to the medicine being administered, who might have cognitive degeneration or impairment such that they forget their dosage, or who might have other unscrupulous motives to access the stash of medicine.

Additional conventional secure pill dispensers are locked and automatically dispense medicine at times deemed to be appropriate for ingesting the medicine. These pill dispensers can be broken, unlocked, or stolen, and the medicine inside accessed in bulk, and/or by individuals not intended to receive the medicine, unbeknownst to authorities or medic al professionals.

### SUMMARY OF THE INVENTION

A “smart”, secure pill dispenser and pill dispensing system can record and report patient behavior as it relates to administration of medicine outside the direct care of a doctor, in order to reduce or prevent medicine abuse, over-use, under-use, and misuse, and to facilitate proper medicine administration to a patient outside the direct supervision of a doctor.

In an embodiment of the invention, a pill dispensing system includes a pill dispenser, which includes a body, a dispensing port in the body to dispense medicine from the body, and a computer processor supported by the body.

In an embodiment, a pill dispensing system includes a dispensing system platform and a pill dispenser configured to communicate with the dispensing system platform. The dispensing system platform includes an administrator module having a central database and an administrator portal configured for system administrator access.

In an embodiment, a computer system for dispensing prescription medication comprises a plurality of computers, each computer of the plurality of computers comprising at least one processor, one or more memories, one or more computer readable storage media having program instructions executable by the computer to perform the program instructions comprising: prompting, by a first of the plurality of computers, an input of an electronic prescription order and a treatment instruction; sending, by the first of the plurality of computers, the prescription order and the treatment instruction to a second computer of the plurality of computers; correlating and recording, by the second of the plurality of computers, a pill dispenser identifier and prescription information; and prompting, by a third computer of the plurality of computers, activation of the pill dispenser.

In an embodiment, a pill dispenser key device includes a body housing a processor, a memory, and a communication element, the processor, the memory, and the communication element configured to communicate a digital key to a pill dispenser.

### BRIEF DESCRIPTION OF THE DRAWINGS

FIG. 1 illustrates a pill dispensing system including a pill dispenser, a dispenser key, and a dispensing system platform, according to an embodiment.

FIG. 2 illustrates the pill dispenser according to the embodiment of FIG. 1.

FIG. 3 schematically illustrates a memory, a communication element, and a processor of a pill dispenser, according to an embodiment.

FIG. 4 illustrates the dispenser key device according to the embodiment of FIG. 1.

FIG. 5 schematically illustrates a processor, a memory, and a communication element, according to an embodiment.

FIG. 6

FIG. 7 schematically illustrates a dispensing system platform, according to an embodiment.

FIG. 8 illustrates an example diagram of a possible data processing environment in which illustrative embodiments may be implemented.

FIG. 9 illustrates internal and external components of the device computer 152 and the server computer, according to an embodiment.

FIGS. 10-16 illustrate some examples of displays or pages of a patient module.

FIGS. 17-19 illustrate some examples of displays or pages of a doctor module.

FIG. 20 is a flow chart showing prescription flow via the pill dispensing system.

### DETAILED DESCRIPTION OF THE INVENTION

In the following description, reference is made to the accompanying drawings that form a part thereof, and in which is shown by way of illustration specific exemplary embodiments in which the present teachings may be practiced. These embodiments are described in sufficient detail to enable those skilled in the art to practice the present teachings and it is to be understood that other embodiments may be utilized and that changes may be made without departing from the scope of the present teachings. The following description is, therefore, merely exemplary.

Disclosed is a tamper-resistant pill dispensing system which can be integrated with business to business electronic medical records (“EMR”) or electronic health records (“EHR”), for use by patients, physicians, insurance companies, pharmaceutical companies, and government agencies. The system aims to reduce prescription drug abuse, patient non-adherence, overdose, under-dose, prescription drug trafficking, and their related effects. The system can interfere with, or break, the cycle that leads to prescription drug abuse and can prevent the development of addiction tendencies in patients by consistently providing only doctor approved dosage amounts to a specific patient. The pill-dispensing system can be used whenever patients are prescribed controlled substances (i.e. Schedule II drugs such as opioids, stimulants, anxiolytics, and antidepressants) or other substances where intensive physician monitoring would be beneficial (e.g., treatment of chronic diseases, treatment of terminal illnesses, treatment of mental health issues, and administration of antibiotics, among others).

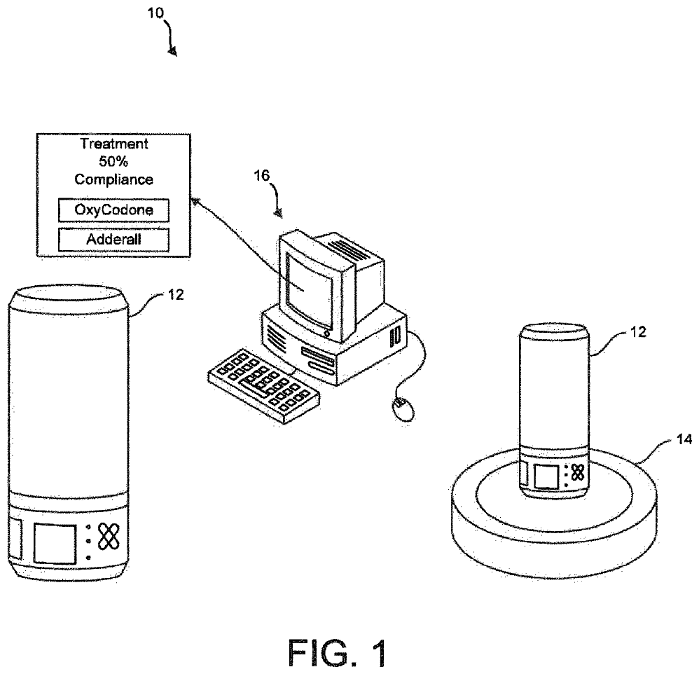

FIG. 1 illustrates a pill dispensing system 10, including a pill dispenser 12, a dispenser key 14, and a dispensing system platform 16, according to an embodiment. The pill dispenser 12 can be filled with medicine by a pharmacist, at the direction of a patient's doctor. The dispenser key 14 can be used to lock and unlock the pill dispenser 12 in order to fill the dispenser 12 with medicine. Doctors, pharmacists, insurance company representatives, patients, or others can interact with the pill dispenser 12 directly, or indirectly by accessing the dispensing system platform 16 via various portals, including a doctor portal, a pharmacist portal, a patient portal, etc.

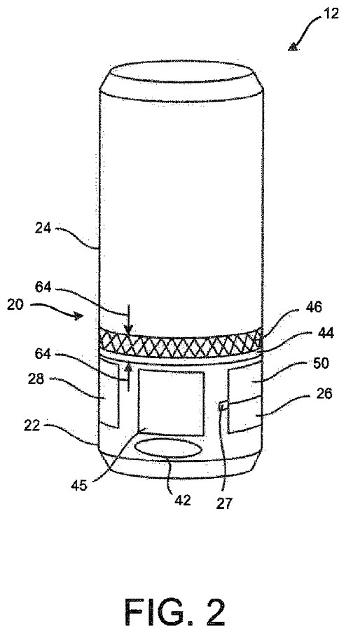

FIG. 2 illustrates the pill dispenser 12. The pill dispenser 12 has a body 20 including a cap 22 and a vial 24, and defining an internal pill storage cavity (not shown) connected to a dispensing port 26. The vial 24 can be sized and shaped to match standard sizes of prescription dram vials, which can facilitate automated processing of the pill dispensers 12. The vial 24 can define and bound a majority of the internal pill storage cavity, with the cap 22 configured to bound a minority of the internal pill storage cavity, or one side thereof. The pill dispenser 12 is configured to dispense from the internal pill storage cavity a number of pills at the dispensing port 26, and/or an amount of medicine, prescribed by the patient's doctor. The pill dispenser 12 has secure access features, such as a biometric identifier 28. The biometric identifier 28 can include one or more of: a fingerprint sensor; voice recognition; a retinal scanner; or other now-known or future-developed identification means. The dispensing port 26 remains closed and locked by a locking element 27 until a time (or range of times) at which the pill dispenser 12 has been programmed to allow a patient to unlock and open the dispensing port 26 and dispense medicine. At this time, the patient can validate his or her identity by activating the biometric identifier 28. In some embodiments, the biometric identifier 28 can also collect biological data of the patient, such as, but not limited to, heart rate, oxygen saturation, and voice characteristics.

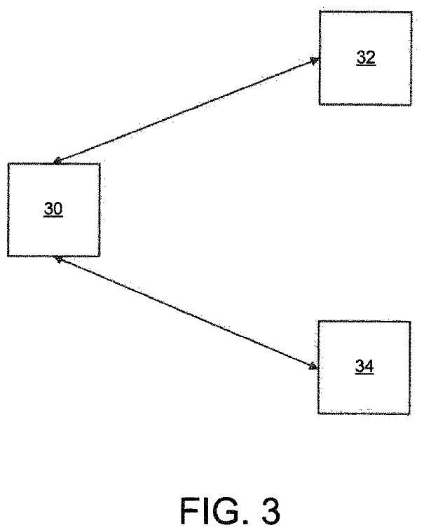

The data collected by the biometric identifier 28 can be stored in a memory 30 and communicated to the dispensing system platform 16 via a communication element 32. This process, the locking and unlocking of the dispenser port 26, and further processes can be controlled by a processor 34. FIG. 3 schematically illustrates the memory 30, the communication element 32, and the processor 34. Each of the memory 30, the communication element 32, and the processor 34 can be housed on or within the cap 22. The communication element 32 can be used to communicate with other devices by direct connection to the device or a shared network, or wirelessly over cellular networks, Bluetooth, or other wireless networks. Each of the electronically-driven functions of the pill dispenser 12 described herein can be controlled by instructions and rules configured on the processer 34 and/or the memory 30, by utilizing the communications element 32 to communicate with and receive instructions from another computing device instantiating at least a portion of the dispensing system platform 16, or by a combination of both.

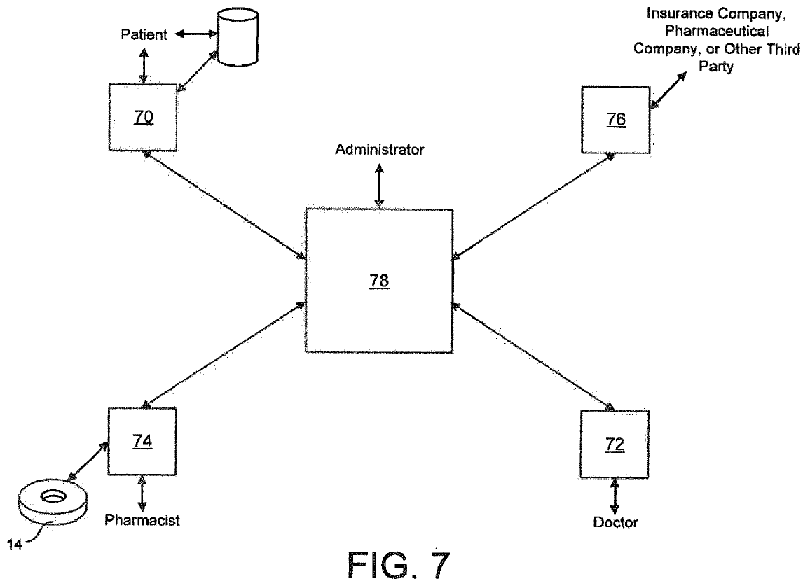

Referring again to FIG. 2, a voice interaction module 40 (see FIG. 7) can be included to provide patients with prescribed dosage information in accordance with the physician's instructions. The voice interaction module can be multilingual. The pill dispenser 12 can include a microphone 42 and a speaker 44, which can both be housed on or in the cap 22, and can both be used with the voice interaction module 40, as well as with a biometric identifier 28 if the biometric identifier utilizes voice identification. The voice interaction module 40 can instruct patients how to properly take their medications, and can provide additional prescription treatment guidance by allowing patients to ask general questions (e.g., “When is my next dosage?”). The voice command can provide patients with the name of the medication, the dosage amount, and directed usage information (e.g., “take with plenty of water”, “take with a meal”, etc.). The voice command feature can be programmed using GPS location from the patient's mobile computing device (e.g., cell phone, tablet, laptop) to function only at specified locations, such as the patient's home. Outside the specified location, the feature can be programmed to remind patients through a sound mode or a vibrate mode to guarantee patient treatment privacy. The voice command, sound, and vibrate features can be programmed by patients to better accommodate to their personal needs.

A display screen 45, such as a touchscreen, can provide all the functionality of the voice interaction module, except in writing rather than sound. The display screen can also be housed in or on the cap 22.

Indication lights 46, which can be light emitting diodes (“LEDs”) or another light source, can illuminate a specified color at a time the patient is scheduled to take a dosage. The indication lights 46 can also be housed in or on the cap 22. While the indication lights 46 can be arranged variously, in the embodiment of FIG. 2 the indication lights are shown as a strip of light emitting diodes encircling an end of the cap 22. To better draw the patient's attention, the indication lights 46 can blink or flicker. The color that the indication lights 46 illuminate can match the color of medication listed in the dispensing system platform 16 in a color coded legend. For example, if a patient is scheduled to take oxycodone and the medication is color coded as blue on the software application, then the indication lights 46 can illuminate blue to indicate it is time to take the medicine.

The pill dispenser 12 can be configured with the ability to record patient conversation and behavioral data, and the ability to report this data to the patient's doctor, as well as to insurance companies or other specified individuals or entities. In this way, early intervention can be provided to prevent misuse, abuse, and addiction to Schedule II prescription medications. Tampering with the pill dispenser 12 can also be detected and reported.

In case of a malfunction that prevents automatic dispensing, an emergency, mechanical dispensing button 50 can unlock and activate the dispenser port 26. The dispensing button 50 can be configured to be depressed or otherwise locked, and pop out or otherwise become active for use only when the pill dispenser 12 sends a signal indicating the pill dispenser 12 is unable to dispense a dosage of medicine. Simultaneously, the indication lights 46 can illuminate and/or the speaker 44 can emit an audible indicator.

In the case of a pill jam in the pill dispenser 12 that would block further pills from being dispensed through the dispenser port 26, the cap 22 can unlock from the vial 24 so that the patient can disconnect the cap 22 from the vial 24. The emergency dispensing button 50 and/or the unlocking of the cap 22 from the vial 24 can be disabled for medicine for which failed administration is non-life threatening, and enabled for medications that are time sensitive, and for which failed administration can be life threatening. Activation of the emergency dispensing button 50 can bypass identity verification by the biometric identifier 28. The emergency dispensing button 50 can also be used as the main dispensing method for patients who are not able to use the biometric identifier 28, who are unable to self-administer medicine, and/or who require third party treatment assistance.

The pill dispenser 12 can be configured such that if a patient not authorized to use the emergency dispensing button 50 as the primary dispensing method uses the emergency dispensing button 50, the pill dispenser 50 and/or the dispensing system platform 16 can require the patient to provide a reason for using the emergency button 50 by selecting an option provided by the dispensing system platform 16. This information can be made available to both physicians and the patient's health insurance provider for further action.

Besides through the dispensing port 26, the only access to the internal cavity (not shown) is by unlocking and removing the cap 22 from the vial 24. The cap 22 can be unlocked from the base 22 electronically by a digital key. The digital key may be provided to select medical professionals, such as pharmacists.

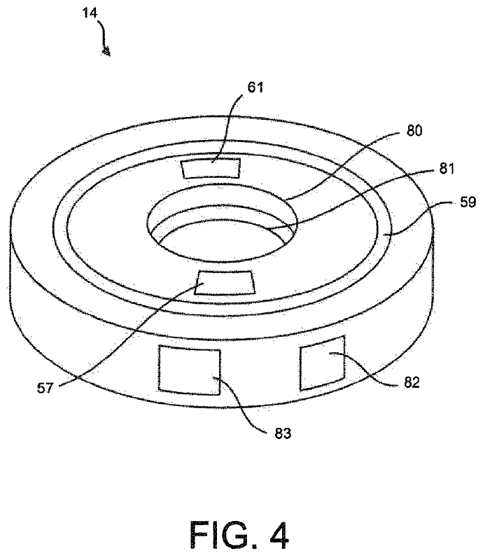

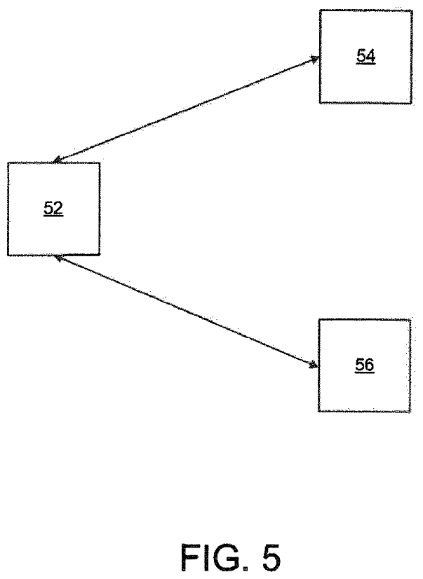

The digital key can be provided by the dispenser key device 14. FIG. 4 illustrates the dispenser key device 14, which can also include a processor 52, a memory 54, and a communication element 56, as illustrated in FIG. 5. The processor 52, memory 54, and communication element 56 provide capability for the dispenser key device 14 to process program instructions, store information, and communicate with a network or other computing device. Accordingly, the dispenser key device 14 can communicate with the pill dispenser 12 and the dispensing system platform 16.

Referring to FIG. 4, the dispenser key device 14 can be configured to receive the pill dispenser 12, such as in receptacle 80, shaped congruently with the cap 22 to receive the cap 22. A single receptacle is shown in the illustrated dispenser key device 14, though any practical number of receptacles 80 can be included in a single dispenser key device 14. Similarly, the shape of the dispenser key device 14 is shown as that generally of a disc, which is not intended to be limiting.

A first scanner 81 can be located in the receptacle 80, positioned such that when the cap 22 is placed in the receptacle 80, the first scanner 81 can read a barcode or other type of code scan-able or readable located on the cap 22 automatically using a scanning device. For simplicity, herein, all types of scan-able or readable codes are referred to as barcodes. Any information associated with the barcode can be stored in the memory 54 and/or communicated to the pill dispenser 12, the dispensing system platform 16, another network used by the pharmacist, or other networks or computers. For example, information to identify and catalog the particular pill dispenser 12 can be collected by the dispenser key device 14 in this manner. Every cap 22 can be assigned a specific and unique alphanumeric code, for example, and any time the cap 22 is placed in the receptacle 80 of the dispenser key device 14, the dispenser key device 14 and the dispensing system platform 16 can know this unique code and precisely which pill dispenser 12 is in the dispenser key device 14.

The dispenser key device 14 can also include a second scanner 82 to read barcodes. The second scanner 82 can be configured to read the same types of barcodes that the first scanner 81 reads, or different types of barcodes. The second scanner 82 is positioned in a location relatively convenient for a pharmacist to scan a barcode affixed (e.g., by the pharmacist when filling a prescription) to a side of the pill dispenser 12. The barcode can correspond to information related to the patient and the prescription, such as but not limited to medicine type and quantity.

The information retrieved by the first scanner 81 or the second scanner 82 can be displayed a display 83. The display 83 can be any now-known or future-developed illuminating screen, such as a touchscreen, an LED, and OLED, etc. The display 83 can also display warnings, advertisements, or notices. The display 83 can display a notice that a new prescription has arrived, and/or display the prescription order, or any other information stored by or received by the dispenser key device 14.

The dispenser key device 14 can be configured such that the digital key, which can be a special code directing the pill dispenser 12 to lock or unlock, or to allow the cap 22 to be unlocked from the vial 24, can be communicated to the pill dispenser 12 as a result of the first scanner 81 reading the barcode on the top of the cap 22. In some embodiments, near field communication can be used to exchange the digital key. In these embodiments, for example, when the pill dispenser 12 is placed within close proximity (e.g., less than six inches) to the dispensing key device 14, the information is shared. In some embodiments, the digital key is not communicated between the pill dispenser 12 and the dispenser key device 14 until an unlocking element 57, such as an unlocking button on the dispenser key device 14 is activated. Referring to FIG. 4, the unlocking button 57 to unlock the pill dispenser 12, or key indicator lights 59, can illuminate one or more colors of light to indicate various statuses. A first color, such as green, can indicate information is ready to be shared between the pill dispenser 12 and the dispenser key device 14. A second color, such as blue, can indicate successful sharing of information between the pill dispenser 12 and the dispenser key device 14. A third color, such as red, can indicate unsuccessful sharing of information between the pill dispenser 12 and the dispenser key device 14.

The digital key can also have a biometric recorder 61, such as a fingerprint scanning pad, which can be used to record a biometric identifier of the patient, such as the patient's fingerprint. The saved biometric identifier can be correlated with unique code of the pill dispenser 12, and both can be transmitted to the pill dispenser 12 and/or the dispensing system platform 16, and saved under a personal treatment chart of the patient.

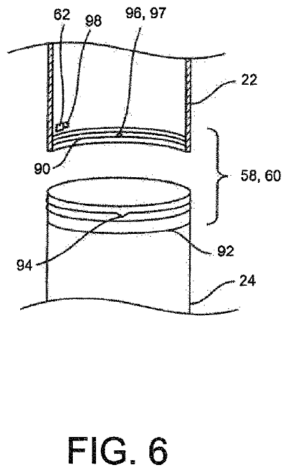

A two-step locking element restricts access to an internal pill storage cavity (not shown). FIG. 6 shows an example of a two-step locking 58 wherein the cap 22 has interior threads 90 and the vial 24 has exterior threads 92. The cap 22 is shown sectioned to reveal the interior threads 90. A primary locking element 60 of the two-step locking element 58 can activate upon connecting the cap 22 with respect to the vial 24. The cap 22 can twist onto the vial 24 in a conventional manner using threads on the cap 22 and the vial 24, or the cap 22 can connect to the vial 24 by other conventional means. The primary locking element 60 can engage before the cap 22 is fully connected. For example, with a twist-on cap, the primary locking element 60 can engage after twisting the cap 22 a predetermined portion leaving room for the cap 22 to be twisted further. The primary locking element 60 can retain the cap 22 on the vial 24, while allowing removal upon applying a threshold removal force, to allow a user to remove the cap manually without difficulty. A first sound or other indicator can designate when the primary locking element 60 is activated or deactivated, to alert the user (i.e., the pharmacist).

In the example shown in FIG. 6, the exterior threads 92 can have a protrusion 94 which can engage a depression 96 in the cap 24 among the interior threads 90. A sensor 98 at the depression 96 can detect engagement of the protrusion 94 with the depression 96, and can generate a signal to the processor 34. The protrusion 94 in the depression 96 can have enough friction to retain the cap 22 on the vial 24, while allowing a reasonable force to twist the cap 22 further on to the vial 24 or off the vial 24.

As a result of the cap 22 being twisted until the primary locking element 60 is activated, the pill dispenser 12 can emit a voice message via the speaker 44 and/or a color coded illumination via the indication lights 46. The voice message can provide a user (i.e., the pharmacist) with a warning message upon activation of the primary locking element 60. An example of a warning message can be, “Please verify or make sure that the prescription was filled properly prior to twisting the cap further to engage the secondary locking mechanism”. In this way, the primary locking element 60 can allow pharmacists to verify if the medication was filled in accordance to the prescription order, and to untwist the cap 22 to open the pill dispenser 12 in case any mistakes are made during the prescription filling process. Each pill dispenser 12 can be provided to the pharmacist with a placebo placed in the internal storage cavity (not shown) so the pharmacist can conveniently test the device.

A secondary locking element 62 of the two-step locking element 58 can be activated by twisting the cap 22 further. Amongst other variations, the secondary locking element 62 can be a mechanically activated lock with an electronic release. A sensor 99 can determine when the cap 22 is fully connected to the vial 24 to trigger engagement of the secondary locking element 62, and a second sound, voice message, or other indicator can designate when the secondary locking element 62 is activated or deactivated. The secondary locking element 62 can be activated when the cap 22 is twisted all the way onto the vial 24. Alignment indicators 64 (see FIG. 2) on the pill dispenser 12, such as, but not limited to arrows, lines, or grooves, can act as guides, or further indicators that the cap 22 is fully connected and secured on the vial 24.

A pharmacist can use the unlocking button 57 to unlock the pill dispenser 12 upon placement of the pill dispenser 12 on the center of the dispenser key device 14. If the pill dispenser 12 needs to be unlocked, the pharmacist can press the unlocking button 57 located on the dispenser key device 14. As a result of being pressed, the unlocking button 57 can illuminate one or more designated colors. When the unlocking button 57 illuminates, the pharmacist can place the desired pill dispenser 12 to be unlocked on the dispenser key device 14. Upon placement of the pill dispenser 12 and successful sending of an unlock signal from the dispenser key device 14, the unlocking button 57 stops illuminating and the key indicator lights 59 can illuminate a designated color, and then dim.

Lock/Unlock signals sent by the dispenser key device 14 to the pill dispenser 12 can constantly change to reduce or prevent hacking of digital keys.

Referring to FIG. 7, doctors, pharmacists, insurance company representatives, patients, or others can interact with the pill dispenser 12 directly, or indirectly by accessing the dispensing system platform 16 via various modules of the dispensing system platform 16, including a patient module 70, a doctor module 72, a pharmacist module 74, and a third-party module 76. Each module 70, 72, 74, 76 can include a portal for direct interaction between the dispensing system platform 16 and a patient, doctor, pharmacist, or insurance company, respectively. Also, each module 70, 72, 74, 76 can communicate with an administrator module 78, which can include a portal for an administrator's access, and a central database of information received from each module 70, 72, 74, 76. The administrator module 78 can exist on a server or server network, and the patient module 70, the doctor module 72, the pharmacist module 74, and the third-party module 76 can each exist on one or more client computing devices. In this way, the dispensing system platform 16 can be distributed over various computing devices connected to a server over a network. The pill dispenser 12 can also be connected to this network.

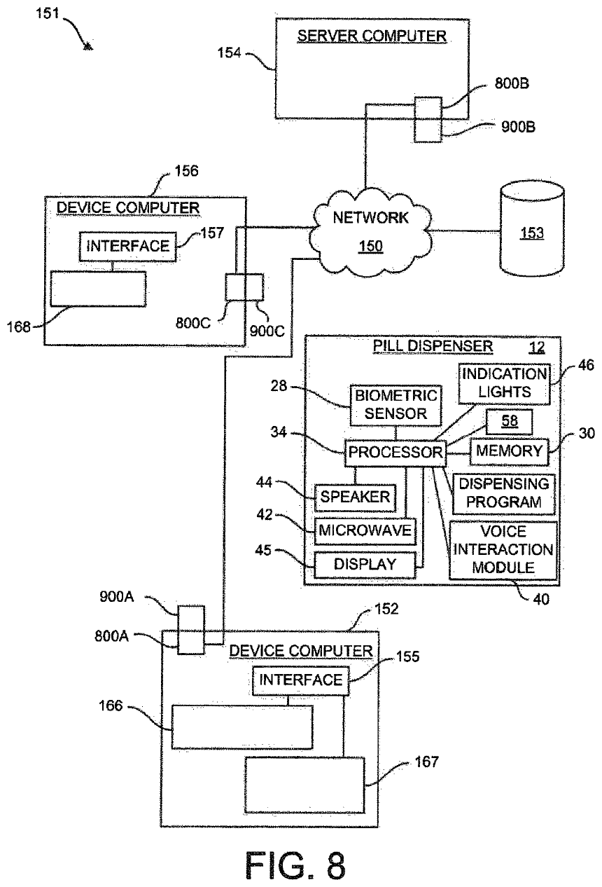

FIG. 8 illustrates an example diagram of a possible data processing environment in which illustrative embodiments may be implemented. It should be appreciated that FIG. 8 is only an example and is not intended to assert or imply any limitation with regard to the environments in which different embodiments may be implemented. Many modifications to the depicted environments may be made.

Referring to FIG. 8, network data processing system 151 is a network of computers in which illustrative embodiments may be implemented. Network data processing system 151 contains network 150, which is the medium used to provide communication links between various devices and computers connected together within network data processing system 151. Network 150 may include connections, such as wired connections, wireless communication links, or fiber optic cables.

In the depicted example, device computer 152, a repository 153, and a server computer 154 connect to network 150. In other example embodiments, network data processing system 151 may include additional client or device computers, storage devices or repositories, server computers, and other devices not shown. The network data processing system may also include the pill dispenser 12, which also is or includes a device computer.

The device computer 152 may contain an interface 155, which may accept commands and data entry from a user. The interface can be, for example, a command line interface, a graphical user interface (GUI), a natural user interface (NUI) or a touch user interface (TUI). The device computer 152 can, for example, include the patient module 70, the doctor module 72, the pharmacist module 74, or the third party module 76. While not shown, it may be desirable to have all or portions of the any one of the modules 70, 72, 74, 76 present on the server computer 154. The device computer 152 includes a set of internal components 800a and a set of external components 900a, further illustrated in FIG. 8.

The server computer 154 includes a set of internal components 800b and a set of external components 900b illustrated in FIG. 8. In the depicted example, server computer 154 provides information, such as boot files, operating system images, and applications to the device computer 152. Server computer 154 can compute the information locally or extract the information from other computers on network 150.

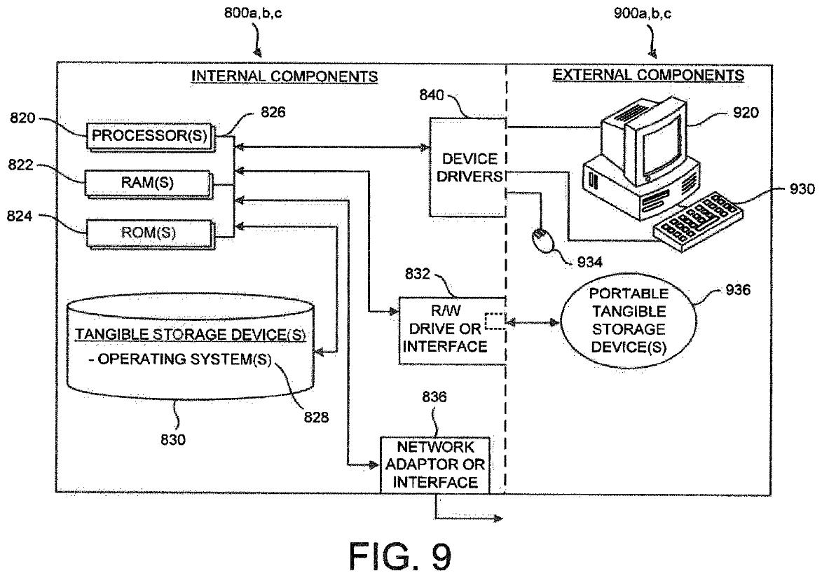

Program code and programs to implement the modules 70, 72, 74, 76, 78 may be stored on at least one of one or more computer-readable tangible storage devices 830 shown in FIG. 8, on at least one of one or more portable computer-readable tangible storage devices 936 as shown in FIG. 8, or on storage unit 153 connected to network 150, or may be downloaded to a device computer 152 or server computer 154, for use. For example, program code and programs such as for the modules 70, 72, 74, 76, 78 may be stored on at least one of one or more storage devices 830 (FIG. 9) on server computer 154 and downloaded to device computer 152 over network 150 for use. Alternatively, server computer 154 can be a web server, and the program code, and programs such as modules 70, 72, 74, 76, 78 may be stored on at least one of the one or more storage devices 830 on server computer 154 and accessed device computer 152. In other example embodiments, the program code, and programs such as for the modules 70, 72, 74, 76, 78 may be stored on at least one of one or more computer-readable storage devices 830 (FIG. 9) on device computer 152 or distributed between two or more servers.

In the depicted example, network data processing system 151 is the Internet with network 150 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 151 also may be implemented as a number of different types of networks, such as, for example, an intranet, local area network (LAN), or a wide area network (WAN). FIG. 8 is intended as an example, and not as an architectural limitation, for the different illustrative embodiments.

FIG. 9 illustrates internal and external components of the device computer 152 and the server computer 154 in which illustrative embodiments may be implemented. In FIG. 9, the device computer 152 and the server computer 154 include respective sets of internal components 800a, 800b and external components 900a, 900b. Each of the sets of internal components 800a, 800b includes one or more processors 820, one or more computer-readable RAMs 822 and one or more computer-readable ROMs 824 on one or more buses 826, and one or more operating systems 828 and one or more computer-readable tangible storage devices 830. The one or more operating systems 828 and programs to implement the modules 70, 72, 74, 76, 78 are stored on one or more of the computer-readable tangible storage devices 830 for execution by one or more of the processors 820 via one or more of the RAMs 822 (which typically include cache memory). In the embodiment illustrated in FIG. 9, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory or any other computer-readable tangible storage device that can store a computer program and digital information.

Each set of internal components 800a, 800b also includes a R/W drive or interface 832 to read from and write to one or more portable computer-readable tangible storage devices 936 such as a CD-ROM, DVD, memory stick, magnetic tape, magnetic disk, optical disk or semiconductor storage device. Programs to implement the modules 70, 72, 74, 76, 78 can be stored on one or more of the portable computer-readable tangible storage devices 936, read via R/W drive or interface 832 and loaded into hard drive 830.

Each set of internal components 800a, 800b also includes a network adapter or interface 836 such as a TCP/IP adapter card. The programs to implement the modules 70, 72, 74, 76, 78 can be downloaded to the device computer 152 and the server computer 154 from an external computer via a network (for example, the Internet, a local area network or other, wide area network) and network adapter or interface 836. From the network adapter or interface 836, programs to implement the modules 70, 72, 74, 76, 78 are loaded into hard drive 830. Programs to implement modules 70, 72, 74, 76, 78 can be downloaded to the server computer 154 from an external computer via a network (for example, the Internet, a local area network or other, wide area network) and network adapter or interface 836. From the network adapter or interface 836, programs to implement the modules 70, 72, 74, 76, 78 are loaded into hard drive 830. The network may comprise copper wires, optical fibers, wireless transmission, routers, firewalls, switches, gateway computers and/or edge servers.

Each of the sets of external components 900a, 900b includes a computer display monitor 920, a keyboard 930, and a computer mouse 934. Each of the sets of internal components 800a, 800b also includes device drivers 840 to interface to computer display monitor 920, keyboard 930 and computer mouse 934. The device drivers 840, R/W drive or interface 832 and network adapter or interface 836 comprise hardware and software (stored in storage device 830 and/or ROM 824).

Programs to implement the modules 70, 72, 74, 76, 78 can be written in various programming languages including low-level, high-level, object-oriented or non object-oriented languages. Alternatively, the functions of the modules 70, 72, 74, 76, 78 can be implemented in whole or in part by computer circuits and other hardware (not shown).

Blockchain technology can be used with the dispensing system platform 16 for security purposes to prevent unauthorized access to patient treatment data, to protect the pill dispenser 12 and the dispensing system platform 16 from hacking. The blockchain technology can allow patients to be alerted of any inappropriate personal treatment health data access, and to have more control over their treatment regime as the patients will have the ability to select who and what level of personal health data can be made accessible by different authorized parties (i.e. primary care physician, specialists, family caregiver, etc).

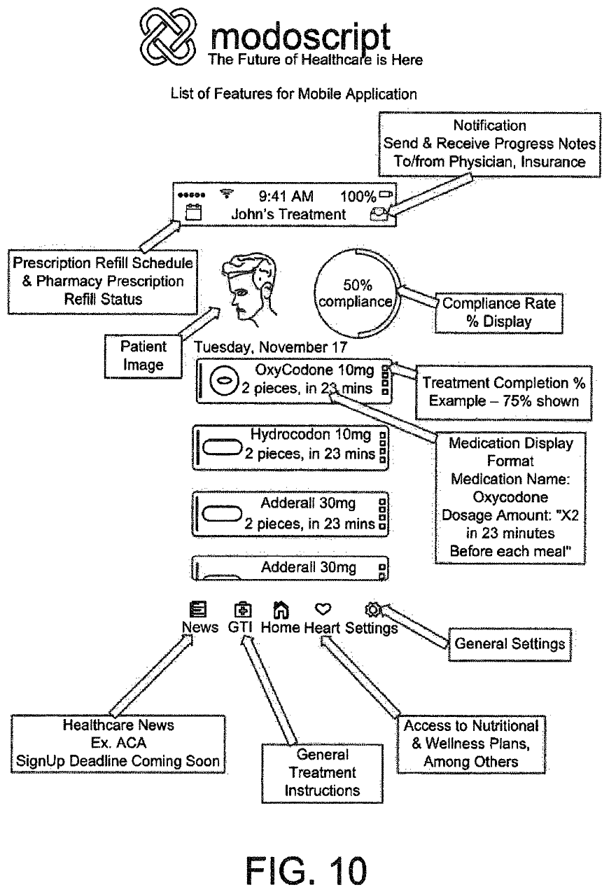

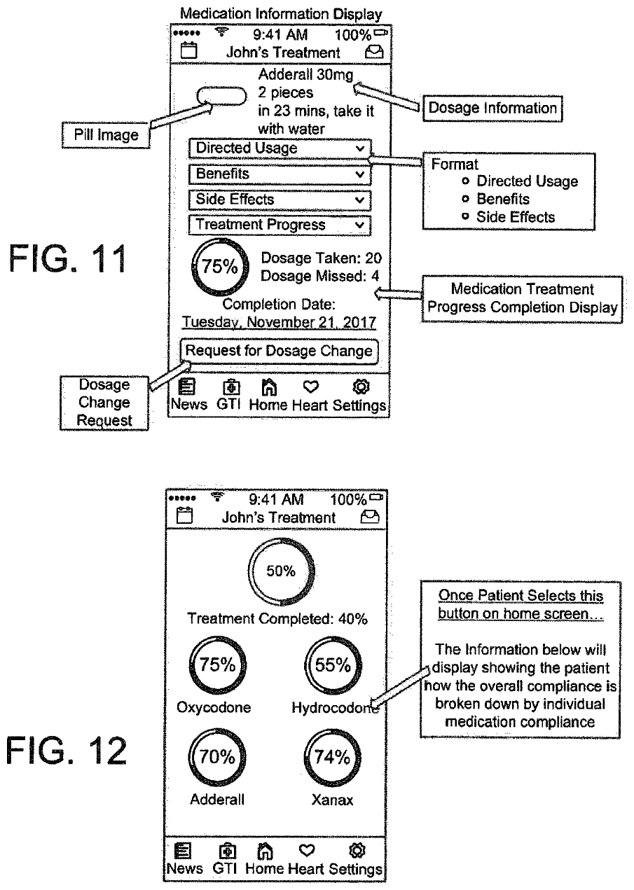

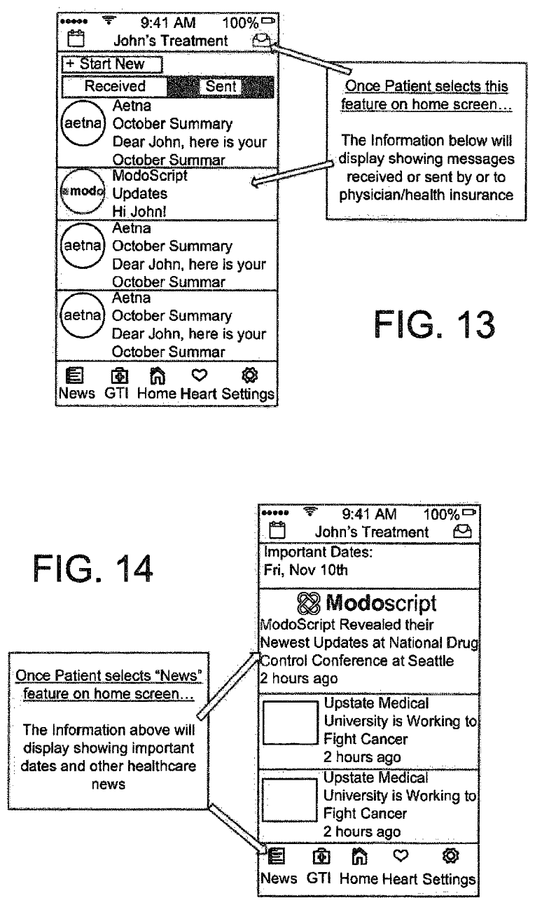

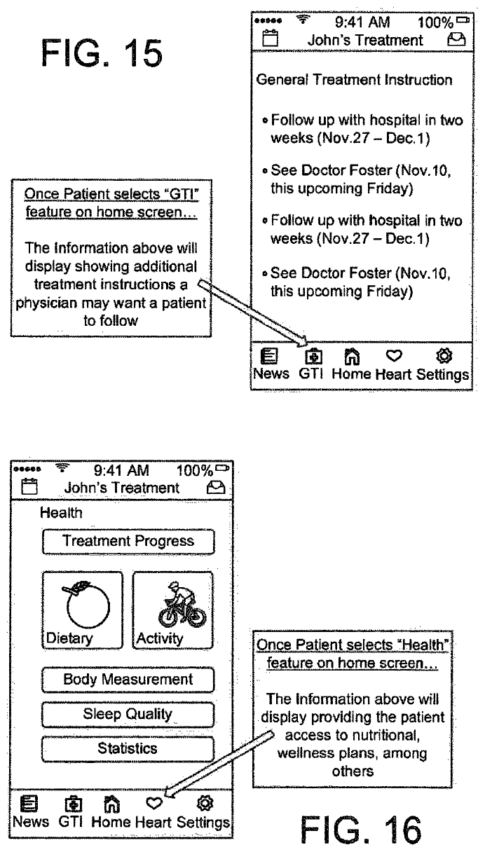

FIGS. 10-16 illustrate some examples of displays or pages of the patient module 70. The patient module 70 can provide patients with pre-programmed general and prescription treatment plans. The application can be structured to educate patients more about their respective treatments by providing them with step by step treatment guidance and educational information (e.g., benefits, side effects, and directed usage) for each medication prescribed. A checklist with any additional treatment instructions that physicians may want their patients to follow can also be provided (e.g., “Please visit Neurologist, Dr. Chin”). Furthermore, via the patient module 70, patients can have access to nutritional and wellness plans and/or can be able to receive push notifications from their physicians regarding topics such as, but not limited to, treatment progress, health insurance providers, and appointment reminders). Upon consent, patients can communicate with their physicians or insurance providers, via messaging or video conference (e.g., physician checking up on a patient's prescription treatment progress).

Additionally, the patient module 70 can allow patients to request dosage changes depending on how the patients react to their prescription treatment plan, which can require approval from their doctors. To ensure patient safety and proper treatment guidance, the patient module 70 can send out automated pop-up treatment progress related questions (e.g. “How are you feeling one week into your prescription treatment plan? A. Better B. The Same C. Worse”) at least but not limited to 3 times a week. Other features to better assist patients can include having access to nutritional and wellness plans to further improve the rate of recovery or resolution of their symptoms, and having access to important healthcare related news that might apply to a patient (e.g., “Flu season is around the corner . . . learn more or schedule an appointment with your primary care physician”). The patient module 70 can also display pop-up pharmaceutical ads every 3^{rd}, 4^{th}, or other number of times a patient accesses the patient module 70.

The pill dispenser 12 can pair via Bluetooth or private Wi-Fi with a patient's smart phone or other computing device running the patient module 70. The pill dispenser 12 can also pair to the patient's mobile device via wireless carrier. Patients without smartphones can access the patient module 70 and its features, such as educational information, progress notes, and dosage change requests, among others, via the internet. Now-known or future-developed security measures can be implemented to assure the correct identity of a patient logging into the patient portal 70 via any computing device.

In cases where a patient fails to take his or her medications as instructed by the prescribing doctor, the pill dispenser 12 can record and send this information through the patient module 70 to the administrator module 78, which can direct that a notification be sent to the cellular device of the patient, the pill dispenser 12, and/or the patient module 70, as well as a text message to the patient's family emergency contact, as an incentive to further combat prescription non-adherence.

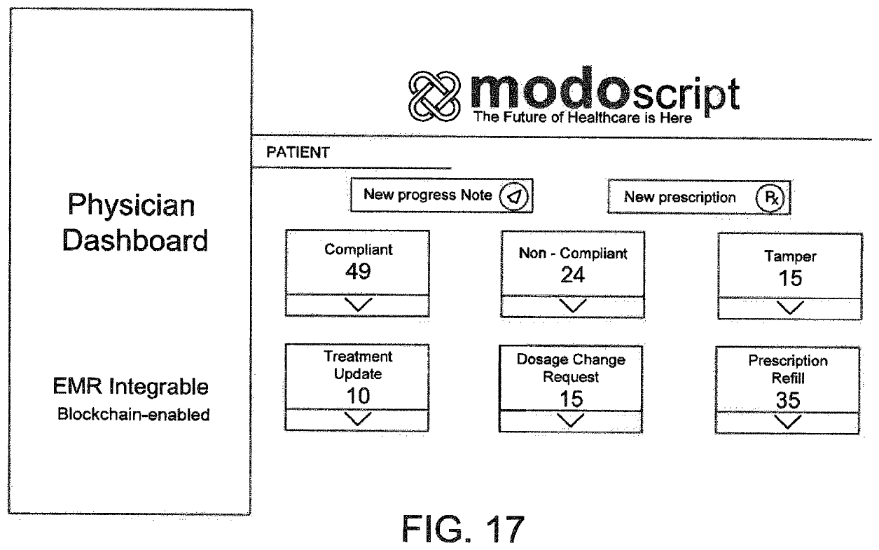

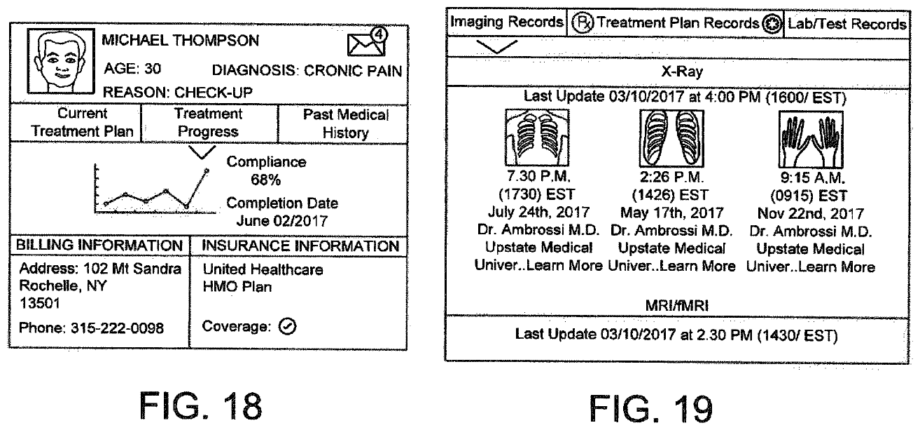

FIGS. 17-19 illustrate some examples of displays or pages of the doctor module 72. The doctor module 72 can be accessible as a stand-alone application running on a computing device, or accessible within the Electronic Medical Records (EMR) system that can provide physicians with live monitoring of information of their patients' treatment progress. Through the doctor module 72, doctors can have access to the number and list of patients who are following their treatments accordingly, patients who have missed multiple dosages or who are not being compliant with the checklist of additional treatment instructions, patients who attempt to tamper with the pill dispensing 12, patients who need to have their treatments revised or updated, and patients who request a dosage change. Because the pill dispenser 12 and the dispensing system platform 16 are able to keep track of how many pills are dispensed at any given time, the doctor module 72 can alert physicians prior to any patients' medications running out to facilitate a smooth prescription refilling or renewal process.

The doctor module 72 can provide doctors with access to patient treatment plan records, imaging records, and lab/test records of any particular patient undergoing or who has undergone a general or prescription treatment plan using the pill dispensing system 10. Authorized personnel (e.g., physician, nurse, physician assistant) can have the ability to upload images or lab/test results to the dispensing system platform 16 through the doctor module 72. The doctor module 72 can also retrieve patient imaging records from EMR using algorithms and store the patient imaging records under the patients' personal treatment charts.

The pharmacist module 74 can provide pharmacists with real-time patient treatment data analytics (medication name, participant name, device identification code (e.g., IQR #), number of pills dispensed, time, date, location, and fingerprint verification) and a list of participants who are compliant, who are not compliant, who attempt to tamper with the pill dispenser 12, and whose medications are running low. The pharmacist module 74 can include a message board with feedback from patients along with additional data collected from surveys through the dispensing system platform 16. The pharmacist module 74 can rate patients based on compliance percentage and can provide pharmaceutical companies with additional clinical trial data analytics.

The third-party module 76 can be used and/or configured for use by a variety of third parties to interact with the pill dispenser 12, the dispenser key device 14, and/or the other parts of the dispensing system platform 16. For example, subject to privacy ethics and laws, health insurance providers can use the third party module 76 to access patient treatment and general patient population data analytics of any medication flowing through the market. Insurance providers can also have access to compliance rates and additional statistics of any individual member or of any desired population. This data can be broken down by state, region, county, town, sex, gender, condition, etc.

In another example, the third party module 76 can be configured for access by a government agency, such as, but not limited to the U.S. Department of Health, NIH, CDC, and FDA. Through the third party module 76, the government agency can access patient treatment data analytics broken down by state, region, county, town, sex, gender, condition, etc. The data can be kept anonymous and restricted from individual treatment data.

Data from all insurance providers and Medicare/Medicaid can be bundled together and available for government agencies to have live statistics of the general flow of any medication, broken down by compliance rates, tampering statistics, population data broken down by age (e.g., missed dosages, tampering, etc.).

In another example, the third party module 76 can provide pharmaceutical companies with real-time access to pharmaceutical supply chain data of the different medications offered to patients to ensure proper protocol is followed, and allow for further improvement to reduce costs. This data can also be used by pharmaceutical companies to buy ad space, and have access to data analytics on consumer outreach via targeted pharmaceutical ads. Medication treatment progress data (e.g., time of dosage, dosage amount dispensed, extra dosages dispensed, tamper alerts, etc.) received for each patient can be saved in the central database of the administrator module 78, and can be accessible to one or more specified pharmaceutical companies through the third party module 76. Medication data provided can be used by pharmaceutical companies, for example, to study the causation of side effects, which can then assist the pharmaceutical companies to develop safer medications for patients.

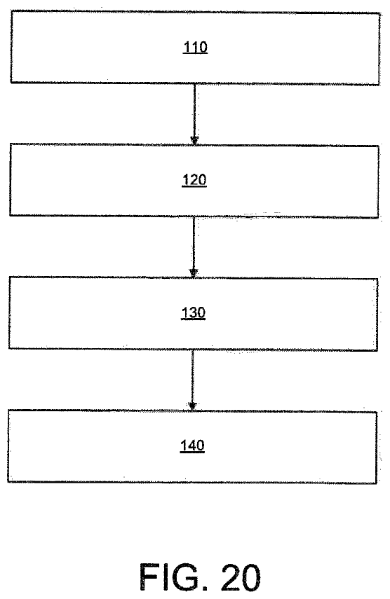

FIG. 20 is a flow chart showing prescription flow via the pill dispensing system 10. In a first step, (Step 110), the pill dispensing system 10 can require electronic prescription order and treatment instructions. These prescription order and treatment instructions can be input through the doctor module 72. At the time the doctor or authorized personnel (e.g., physician's assistant, nurse) sends an electronic script to a pharmacy, he or she can be prompted to answer one or more simple questions via the doctor module 72. The questions can be provided using drag down or check boxes, or another format, to program the desired treatment plan. Some examples of questions include:
1. Length of treatment plan (e.g., 30 days, 60 days, etc.);
2. Method of dispensing upon biometric sensor verification, dispensing button, or both;
3. Dosage amount to be dispensed (e.g., 1 pill, 2 pills, etc.);
4. Desired time interval between dosages (e.g., 4 hours, 8 hours);
5. Number of extra dosages or pills allowed (1, 2, 3, etc.);
6. In how long would you like to check back on [patient name]'s prescription treatment plan (5 days, 1 week, 2 weeks, etc.); and
7. General Treatment Plan Instructions—enter instructions manually or fill-in using check box, or drop down options.

After this information is submitted, this information and the prescription information can be saved on the administrator module 78 and the electronic script can be sent to a designated pharmacy, to the pharmacist module 74 residing on a computing device or computing network at the designated pharmacy, to the dispenser key device 14, and/or to the auto-refilling machine used for regular prescription filling purposes.

In step 120, the pill dispensing system 10 can facilitate filling of the pill dispenser 12 according to the prescription. The prescription order and treatment instructions can be conveyed to the pharmacist module 74, to the dispenser key device 14, or otherwise to the pharmacy, and the pill dispenser 12 can be filled, either automatically by a refilling machine or manually. An auto-refilling machine can fill the vial 24, or the vial 24 can be filled manually by a pharmacist based on the prescription order information received and displayed by, for example, the display 83 of the dispenser key 14.

At a “verification and batching” stage, the cap 22 can be connected to (e.g., twisted onto) the filled vial 24 until the primary locking element 60 is activated. This activation of the primary locking element 60 can activate the pill dispenser 12 to instruct the pharmacist via a pre-programmed voice message (e.g. “Please verify or make sure that the prescription is filled properly prior to twisting the cap onto the secondary locking mechanism”), and to flash a color-coded array of colors (e.g., blue, white, and red).

Upon completion of “verification and batching”, the cap 22 can be connected (e.g., twisted) further to engage the secondary locking element 62. As discussed above, when the secondary locking element 62 is engaged, a second indicator (e.g., a sound or light, such as but not limited to a “click” or LED illuminating) is activated. Additionally, the alignment indicators on the cap 22 and the vial 24 can be aligned when the secondary locking element 62 is engaged.

In step 130, the pill dispensing system 10 can require activating the pill dispenser 12 for patient use. The pill dispensing system 10 can accept input indicating that the filled pill dispenser 12 is picked by or delivered to the patient. The pharmacist can enter this information to the pill dispenser 10, the dispenser key device 10, the pharmacist module 74, or the pharmacist's separate system if it is accessible to the pill dispensing system 10. As a result of the pill dispensing system 10 receiving the information that the filled pill dispenser 12 is picked up by or delivered to the patient, the pill dispensing system 10 can create an access code for the patient to have access to his or her prescription treatment plan. The access code can be sent to the patient via text, email, call, or other means. This access code can be required only for first-time patients, or for first-time and returning patients. In some embodiments, returning patients can have automatic access to their treatment plans through the patient portal 70 and/or the pill dispenser 12. In these embodiments, it may still be the case that attempts to change the treatment plan, such as requesting a dosage change, will require a secret pin, password, or fingerprint for patient identity verification purposes.

The patient can be required to download the patient module 70 in order to use the pill dispenser 12. The patient module 70 and/or the pill dispenser 12 can require the patient to provide a social security number, and/or a security code which can be provided to the patient by email or mobile device. Alternatively, other now-known or future-developed security features to verify the patient identify can be implemented. Proper identity verification can grant the patient access to the general and prescription treatment plan information for the patient.

The patient can then have the opportunity to select a preferred language (English, Spanish, French . . . ) for the course of the treatment plan, applying that choice to the patient module 70. The patient module 70 can inform the pill dispenser 12 (or vice versa) to also communicate with the patient in the chosen language. General treatment plan instructions can remain in the same language submitted by the doctor or authorized personnel, when different, or both languages can be included. For instance, if the patient selects Spanish as the primary language of choice, at the time the patient is instructed by the patient module to biometrically verify the patient's identity, upon successful verification, the pill dispenser 12 can acknowledge the language choice. For example, the pill dispenser 12 can say, “Huella Digital Guardada”.

The patient module 70 or the pill dispenser 12 can also direct the patient to record biometric information. For example, the patient module 70 can direct the patient to record a fingerprint by placing a thumb on a fingerprint reader located on the pill dispenser 12. The pill dispenser 12 can indicate successful biometric information recording. For example, the pill dispenser 12 can play or generate a voice message saying something like, “Fingerprint Recorded”, and/or the indicator lights 46 can illuminate a specified color or color pattern. If the biometric information is not successfully recorded, the indicator lights 46 can illuminate a different specified color or color pattern, and/or a different voice message can be generated, such as, “Please try again”. Alternatively, the dispenser key device 14 can be required and/or used to record the biometric information using the biometric recorder 61. This alternative can facilitate avoiding instances where an improper or unintended person records biometric information because it enable a pharmacist to observe and control the biometric recording.

Upon successful biometric information recording, patients can be required by at least one of the pill dispenser 12, the patient module 70, or the dispenser key device 14 to select when they would like their prescription treatment plan to start, whether it is in 30-min, 1 hour, 2 hours, 1 day, etc. after picking up their prescription. Failure to select a prescription treatment start time can cause the dispensing system platform 16 to send notifications to the patient's phone and/or email, or other patient accounts.

Upon completing the registration and set up steps, patients can be given access to their general and prescription treatment plan, and the treatment plan can start alerting patients, depending the prescription treatment start time selected by patients.

Notably, the pill dispenser 12 can be configured to perform any function described herein above as being performed by the patient module 70.

In step 140 the pill dispensing system 10 can facilitate refilling of prescriptions. The dispensing system platform 16 can alert the patient via the patient module and/or the pill dispenser 12, via text message, via email, or other form of communication when a refill script is sent to the pharmacy. From there, based on the phone's location, patients can have the option to select a pharmacy of their choice around their current location or select the primary pharmacy of choice that they have saved on their prescription treatment chart.

For prescription refilling purposes using mail order service, at the time patients originally receive their prescription order, along with the prescription order, there can be a prepaid bubble mailer padded envelope which patients can use to package the used pill dispenser 12 for return shipping or transportation. As a result of the dispensing system platform 16 receiving a notification that the used pill dispenser 12 is returned, the insurance provider can receive a partial reimbursement for the pill dispenser 12.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A pill dispensing system comprising:
a pill dispenser comprising:
a body;
a dispensing port in the body to dispense medicine from the body; and
a computer processor supported by the body.

2. The pill dispensing system of claim 1, wherein the body comprises a cap, the cap configured to connect to a pill vial.

3. The pill dispensing system of claim 2, wherein the cap houses the computer processor and the dispensing port.

4. The pill dispensing system of claim 2, wherein the body further comprises the pill vial.

5. The pill dispensing system of claim 4, wherein the pill vial is a hollow cylinder.

6. The pill dispensing system of claim 1, further comprising a communication element configured to facilitate communication between the computer processor and a computing network or a computing device.

7. The pill dispensing system of claim 1, further comprising a biometric sensor to verify an identity of a user, the biometric sensor in communication with the computer processor.

8. The pill dispensing system of claim 1, further comprising indication lights supported by the body, the indication lights in communication with the computer processor.

9. The pill dispensing system of claim 1, further comprising a speaker and a microphone, each supported by the body, each in communication with the computer processor.

10. The pill dispensing system of claim 1, further comprising an emergency mechanical dispensing element configured to dispense medicine as a result of mechanical actuation.

11. The pill dispensing system of claim 1, further comprising an electronic display screen.

12. The pill dispensing system of claim 2, further comprising a two-step locking element including a primary locking element and a secondary locking element, the primary locking element and the secondary locking element configured to lock the cap to the pill vial.

13. The pill dispensing system of claim 12, further comprising an indicator, wherein the computer processor is configured to activate the indicator upon engagement of the primary locking element, and the primary locking element is configured to be unlocked manually.

14. The pill dispensing system of claim 12, wherein the secondary locking element is configured to be unlocked with a key.

15. The pill dispensing system of claim 1, further comprising a dispenser key device configured to communicate with the pill dispenser to unlock the cap from the vial or to enable the cap to be unlocked from the vial.

16. The pill dispensing system of claim 1, further comprising a dispensing system platform including an administrator module having a central database and an administrator portal configured for system administrator access, the administrator module configured to communicate with the pill dispenser.

17. A pill dispensing system comprising:
a dispensing system platform including an administrator module having a central database and an administrator portal configured for system administrator access; and
a pill dispenser configured to communicate with the dispensing system platform.

18. The pill dispensing system of claim 17, wherein the dispensing system platform further comprises a patient module having a patient portal configured for patient access.

19. The pill dispensing system of claim 18, wherein the patient module is configured to grant a patient access to a prescription treatment plan for the patient.

20. The pill dispensing system of claim 17, wherein the dispensing system platform further comprises a doctor module having a doctor portal configured for doctor access.

21. The pill dispensing system of claim 20, wherein the doctor module is configured for input of a prescription treatment plan for a patient.

22. The pill dispensing system of claim 17, wherein the pill dispenser includes a vial and a cap, and the pill dispensing system further comprises a dispenser key device configured to communicate with the pill dispenser to unlock the cap from the vial or to enable the cap to be unlocked from the vial.

23. A computer system for dispensing prescription medication comprising a plurality of computers, each computer of the plurality of computers comprising at least one processor, one or more memories, one or more computer readable storage media having program instructions executable by the computer to perform the program instructions comprising:
prompting, by a first of the plurality of computers, an input of an electronic prescription order and a treatment instruction;
sending, by the first of the plurality of computers, the prescription order and the treatment instruction to a second computer of the plurality of computers;
correlating and recording, by the second of the plurality of computers, a pill dispenser identifier and prescription information; and
prompting, by a third computer of the plurality of computers, activation of the pill dispenser.

24. The computer system of claim 23, wherein prompting activation includes prompting, by the third computer, input of security information.

25. The computer system of claim 23, wherein prompting activation includes prompting, by one of the second computer and the third computer, sensing of biometric information of a user.

26. A pill dispenser key device comprising:
a body housing a processor, a memory, and a communication element, the processor, the memory, and the communication element configured to communicate a digital key to a pill dispenser.

27. The pill dispenser key device of claim 26, further comprising a receptacle to receive the pill dispenser.

28. The pill dispenser key device of claim 27, further comprising a code scanner in the receptacle to scan a code on the pill dispenser corresponding to information identifying the pill dispenser.

29. The pill dispenser key device of claim 26, further comprising a barcode scanner to scan a code on the pill dispenser corresponding to prescription information.

30. The pill dispenser key device of claim 26, further comprising status indicators to indicate the status of communication between the pill dispenser key device and the pill dispenser.

31. The pill dispenser key device of claim 26, further comprising an unlocking element to transmit a signal to the pill dispenser to enable the pill dispenser to be unlocked and opened.

32. The pill dispenser key device of claim 26, further comprising a biometric recorder to record biometric information of a pill dispenser user.

33. The pill dispenser key device of claim 26, further comprising an electronic display screen.
